(19) United States
(12) Patent Application Publication
(10) Pub. No.: US 2009/0285862 A1
(43) Pub. Date: Nov. 19, 2009

(54) METHODS AND COMPOSITION FOR TREATMENT OF INFLAMMATORY PAIN

(75) Inventor: Colin Stanley Goodchild, Malvern (AU)

Correspondence Address: SEED INTELLECTUAL PROPERTY LAW GROUP PLLC, 701 FIFTH AVE, SUITE 5400, SEATTLE, WA, 98104, US

(73) Assignee: CNSBio PTY LTD., Melbourne (AU)

(21) Appl. No.: 12/299,294

(22) PCT Filed: May 3, 2007
(86) PCT No.: PCT/AU2007/000587

§ 371 Date: Aug. 5, 2009

Related U.S. Application Data
(60) Provisional application No. 60/797,326, filed on May 3, 2006.

(51) Int. Cl. A61K 9/00; A61K 31/535; A61K 31/44; A61K 31/27

(52) U.S. Cl. 424/400; 514/230.8; 514/353; 514/479

(57) ABSTRACT

The present invention relates to the field of pain management, and in particular, the management of inflammatory pain without inducing overt sedation. The present invention features compositions and treatments for inflammatory pain comprising the administration of an amount of an neurokinin (NK) antagonist in combination with a neuronal excitation inhibitor.

### FIELD

The present invention relates generally to the field of pain management, and in particular, the management of inflammatory pain, without inducing overt sedation. More particularly, the present invention provides methods, protocols, compositions and devices which treat, alleviate, prevent, diminish or otherwise ameliorate the symptoms of inflammatory pain.

### BACKGROUND

Bibliographical details of references provided in the subject specification are listed at the end of the specification.

Reference to any prior art in this specification is not, and should not be taken as, an acknowledgment or any form of suggestion that this prior art forms part of the common general knowledge in any country.

Inflammatory pain is precipitated by an insult to the integrity of tissues at a cellular level. It can be associated with penetration wounds, burns, extreme cold, fractures, arthritis, autoimmune conditions, excessive stretching, infections and vasoconstriction. Multiple chemical factors mediate the inflammatory process either directly affecting nociceptors or by sensitising them to touch or movement.

Nonsteroidal anti-inflammatory drugs (NSAIDS) are the most efficacious and most commonly used treatment for inflammatory pain. They are all aimed at inhibiting prostaglandin production through cyclooxygenase inhibition. The benefit they provide is pain amelioration, but the risks are well known and include unwanted gastrointestinal effects in addition to adverse affects on the skin, kidneys, liver and blood forming organs. Side effects associated with NSAIDS include gastropathy, hypertension, kidney damage, increased risk of heart attack and stroke, heartburn, ulcers and gastrointestinal bleeding, allergic reactions and other side effects.

Problems with the first generation of NSAIDS have mostly been related to side effects caused by inhibiting COX1 as opposed to COX2. COX1 is involved in vegetative and restorative activity of tissues, while COX2 is involved in inflammatory pain. For this reason, the focus has been on the COX2 selective anti-inflammatory agents. The newer cyclooxygenase specific inhibitors have a better safety profile because they are weak inhibitors of COX1. The release of COX2 specific anti-inflammatory drugs has lowered risks somewhat. However, several COX2 specific anti-inflammatory drugs, including Bextra and Vioxx are no longer sold due to their adverse effects For example, Bextra has been linked to increased risk of rare and serious skin conditions and increased cardiovascular risk. Vioxx has been linked to increased risk of cardiovascular side effects. Further, Celebrex has been linked to increased risk of heart attacks and strokes and is placed “black boxed” by the TGA. The risk of serious side effects and mortality of NSAIDS can be significant and accordingly many of the people who have inflammatory pain and require long term treatment are unable to safely use NSAIDS.

Steroids, and in particular corticosteroids, have also been utilised in the treatment of inflammatory pain. The use of corticosteroids can be highly effective especially when delivered to the site of the inflammation. Nevertheless, the frequent use of such medication can cause serious side effects including osteoporosis, disruption of hypophyseal hypothalamic axis, high blood pressure, elevated pressure in the eyes, fluid retention and weight gain. Long term use has been linked to cataracts, high blood sugar levels, increased risk of infection, muscle weakness, osteoporosis and slower wound healing.

Finally, other compounds such as chondrotin sulfate, diacerein and glucosamine sulfate have been shown to have some beneficial effects in ameliorating inflammatory pain associated with degeneration of joint tissues, but these compounds act slowly and are less effective in treating inflammatory pain than NSAIDS.

There is a need to develop a safe, efficacious short-term and long-term treatment of inflammatory pain.

### SUMMARY

Throughout the specification and the claims which follow, unless the context requires otherwise, the word “comprise”, and variations such as “comprises” and “comprising” will be understood to imply the inclusion of a stated integer or step or group of integers or steps but not the exclusion of any other integer or step or group of integers or steps.

Methods and compositions which treat, alleviate, prevent, diminish or otherwise ameliorate the symptoms associated with inflammatory pain in a subject are provided. Reference to “inflammatory pain” includes the pain associated with tissue injury and the resulting inflammatory processes. In particular, a method is contemplated for inducing an analgesic response to inflammatory pain without inducing sedation in a mammal comprising administering to the mammal an amount of an neurokinin (NK) antagonist in combination with a neuronal excitation inhibitor, which combination is effective in reducing the level of or otherwise ameliorating the sensation of pain associated with inflammatory processes. Conveniently, the level of analegesia obtained or absence of sedation using the combination of an NK antagonist and neural excitation inhibitor is greater than the level achieved if either one is used alone.

The term “sedation” includes overt sedation.

As used herein, an NK antagonist is defined as any compound which inhibits, decreases or blocks or otherwise impairs the activity of neurokinin 1 (NK1), neurokinin 2 (NK2) or substance P. Such compounds may either act by directly interacting with NK1, NK2 or substance P or may be selective for any of the target receptors for these compounds, such as NK1, NK2 or NK3.

Examples of NK antagonists provided herein and in one particular embodiment are NK1 antagonists. In other particular embodiments, the NK antagonists are NK2 or NK3 antagonists.

Another aspect provides a method of inducing an analgesic response in a mammal suffering inflammatory pain without inducing sedation by administering to the mammal one or more of an NK antagonist concurrently, separately or sequentially with respect to one or more analgesic compounds selected from the list below of compounds which inhibit or decrease neuronal excitation. Compounds which decrease or inhibit neuronal excitation function by reducing, decreasing or blocking pain signals being transmitted to the brain. Herein, these compounds will be referred to as “neuronal excitation blockers”, “excitation blockers”, “neuronal excitation inhibitor” and “antagonists of neuronal excitation”. Such compounds include, without being limited to flupirtine or a pharmaceutically acceptable salt, derivative, homolog or analog thereof, retigabine, compounds that cause opening of neuronal potassium channels; sodium channel blockers; a modulator of CB2 receptors; a modulator of TRPV1 receptors; a local anaesthetic; opioids; neurosteroids; alpha 2 adrenoceptor antagonists; NSAIDS; NMDA antagonists and calcium channel antagonists. The NK antagonist and the neuronal excitation inhibitor are administered in an amount effective to reduce the symptoms of inflammatory pain. Such an effective amount is considered a synergistic effective amount. In addition, a subject may also be specifically selected on the basis of the type of pain and have a selection step for a particular patient or subject forms an aspect of the present invention.

In one aspect, the neuronal excitation inhibitor is an opioid, such as but not limited to fentanyl, oxycodone, codeine, dihydrocodeine, dihydrocodeinone enol acetate, morphine, desomorphine, apomorphine, diamorphine, pethidine, methadone, dextropropoxyphene, pentazocine, dextromoramide, oxymorphone, hydromorphone, dihydromorphine, noscapine, papverine, papvereturn, alfentanil, buprenorphine and tramadol and pharmaceutically acceptable salts, derivatives, homologs or analogs thereof as well as opioid agonists.

Yet another aspect relates to the use of one or more NK antagonists in combination with flupirtine or a pharmaceutically acceptable salt, derivative, homolog or analog thereof in the manufacture of a medicament for inducing an analgesic response in the treatment of inflammatory pain, without inducing overt sedation.

A further aspect relates to the use of one or more NK antagonists and a neuronal excitation inhibitor, such as flupirtine or a pharmaceutically acceptable salt, derivative, homolog or analog thereof, in the manufacture of one or more separate or combined medicaments for inducing analgesia in response to inflammatory pain. Preferably, the analgesia is induced without overt sedation. In one embodiment the NK antagonist is specific for the NK1 receptor and is combined with a neuronal excitation inhibitor such as flupirtine or retigabine.

Even yet another aspect is directed to the use of one or more NK antagonists and one or more sodium channel blockers in the manufacture of a medicament for inducing analgesia in response to inflammatory pain, preferably, without overt sedation. Sodium channel blockers include without being limited to lamotrogine and mexilentine or a pharmaceutically acceptable salt, derivative, homolog or analog thereof.

In addition, the NK antagonist may be used in combination with one or more local anaesthetics such as but not limited to lignocaine, bupivacaine, ropivacaine, and procaine tetracaine or a pharmaceutically acceptable salt, derivative, homolog or analog thereof. Such a combination is proposed to induce analgesia in response to inflammatory pain, preferably without overt sedation.

Furthermore, the NK antagonist may be used in combination with one or more modulators of TRPV1 receptors, such as but not limited to capsaicin, capsazepine, Nb-VNA, Nv-VNA, SB-705498 and anadamide or a pharmaceutically acceptable salt, derivative, homolog or analog thereof. Such a combination is proposed to induce analgesia in response to inflammatory pain, preferably without overt sedation.

Still further, the NK antagonist may be used in combination with one or more modulators of CB2 receptors such as but not limited to SR144528, AM630 and anadamide or a pharmaceutically acceptable salt, derivative, homolog or analog thereof. Such a combination is proposed to induce analgesia in response to inflammatory pain without inducing overt sedation.

Reference to a “neuronal excitation inhibitor” may also include a sodium channel blocker, a local anaesthetic, a modulator of TRPV1 receptor and/or modulator of CB2 receptor. Equally, a sodium channel blocker, a local anaesthetic, a modulator of TRPV1 receptor and/or modulator of CB2 receptor may also be a neuronal excitation inhibitor.

A delivery system is also provided for inducing analgesia in response to inflammatory pain without inducing overt sedation in a mammal comprising an NK antagonist and a compound which decreases or inhibits neuronal excitation or a pharmaceutically acceptable salt, derivative, homolog or analog thereof. In one aspect the NK antagonist of choice is selected from one or more of Aprepitant, Lanpepitant, CP-99,994, SDZ NKT 343, Ezlopitant, CP-96345, CP-99994, CP-122721, MK-869, GR 205171. RP 67580, Dapitant, Lanepitant, Noloitanium, Sarefutant, Casopitant and/or Vestipitant. The delivery system may, for example, be in the form of a cream or injectable, slow or controlled release injectables, sustained release or slow release formulation, or a tamper proof formulation, or a pharmaceutical formulation or coated onto a stent, catheter or other mechanical device designed for use in a medical procedure.

The compounds according to the present invention may be administered, inter alia, orally, transmucosally, rectally including via suppository, subcutaneously, intravenously, intramuscularly, intraperitoneally, intragastrically, intranasally, intrathecally, transdermally or intestinally or injected into a joint. In particularly preferred forms of the present invention, the compounds are orally or transdermally administered.

Examples of inflammatory pain include, without being limited to rheumatoid arthritis, osteo-arthritis, psoriatic arthropathy, arthritis associated with other inflammatory and autoimmune conditions, degenerative conditions such as back strain and mechanical back pain or disc disease, post-operative pain, pain from an injury such as a soft tissue bruise or strained ligament or broken bone, abscess or cellulitis, fibrositis or myositis.

Inflammatory pain is often associated with inflammatory diseases. As used herein “inflammatory diseases and disorders” encompass those disease and disorders, which result in one or more inflammatory response symptoms such as redness, swelling, pain and a feeling of heat in certain areas. Inflammatory pain is often associated with the following diseases: acne, angina, arthritis, aspiration pneumonia, disease, empyema, gastroenteritis, inflammation, intestinal flu, , necrotizing enterocolitis (NEC), pelvic inflammatory disease (PID), pharyngitis, pleurisy, raw throat, redness, rubor, sore throat, stomach flu and urinary tract infections, Chronic Inflammatory Demyelinating Polyneuropathy, Chronic Inflammatory Demyelinating Polyradiculoneuropathy and post-operative pain. Accordingly, the compositions and methods of the present invention ameliorate or decrease or prevent or treat the pain associated with inflammatory processes without inducing overt sedation.

Methods and compositions are provided herein for use in treating inflammatory pain without inducing overt sedation. As used herein, the phrase “without causing overt sedation” means inducing an analgesic effect without causing significant cognitive or general impairment of nervous system function (such as attention or wakefulness). Such effects on cognition leads to a change in the measurement that leads to an erroneous conclusion about the drug combination causing analgesia.

In one aspect, the NK antagonist is combined with flupirtine or pharmaceutically acceptable salt, derivative, homolog or analog thereof. The flupirtine is administered at a dose of between about 0.5 mg/kg and about 20 mg/kg, at intervals of between about 1 hour and about 50 hours and may be administered prior to, simultaneously with or following the NK antagonist.

In a particular embodiment, the mammal is a human. The subject or a group of subjects may be selected on the basis of the type of pain experienced. The “type” of pain may also be subjectively determined based on symptoms described by the subject. Hence, a therapeutic protocol is contemplated which comprises selecting a subject on the basis of symptoms of pain and administering to the subject an NK antagonist and a neuronal excitation inhibitor wherein the treatment does not cause overt sedation.

A further aspect provides a system for the controlled release of an active compound selected from an NK antagonist and a neuronal excitation inhibitor, wherein the system comprises:
(a) a deposit-core comprising an effective amount of a first active compound and having defined geometric form, and
(b) a support-platform applied to the deposit-core, wherein the support-platform contains a second active compound, and at least one compound selected from the group consisting of:
   (i) a polymeric material which swells on contact with water or aqueous liquids and a gellable polymeric material wherein the ratio of the swellable polymeric material to the gellable polymeric material is in the range 1:9 to 9:1, and
   (ii) a single polymeric material having both swelling and gelling properties, and wherein the support-platform is an elastic support applied to the deposit-core so that it partially covers the surface of the deposit-core and follows changes due to hydration of the deposit-core and is slowly soluble and/or slowly gellable in aqueous fluids.

As used herein, the first active compound is one of (i) one or more NK antagonists or (ii) one or more neuronal excitation inhibitors. The second active compound may be (i) or (ii) above.

In another aspect, a system is described for the controlled release for an NK antagonist and a neuronal excitation inhibitor where the system comprises:
(a) a deposit-core comprising an effective amount of the NK antagonist and the neuronal excitation inhibitor; and
(b) a support platform applied to the deposit-core, the support platform comprising at least one compound selected from the group consisting of:
   (i) a polymeric material which swells on contact with water or aqueous liquids and a gellable polymeric material wherein the ratio of the swellable polymeric material to the gellable polymeric material is in the range 1:9 to 9:1, and
   (ii) a single polymeric material having both swelling and gelling properties, and wherein the support-platform is an elastic support applied to the deposit-core so that it partially covers the surface of the deposit-core and follows changes due to hydration of the deposit-core and is slowly soluble and/or slowly gellable in aqueous fluids.

### BRIEF DESCRIPTION OF THE FIGURES

FIG. 1 is a graphical representation of a pain protocol used for testing the efficacy of the compositions of the present invention.

FIG. 2 is a graphical representation of an aprepitant dose response curve compared with saline control and GABAPentin 50 mg/kg in reversal of carrageenan-induced allodynia assessed with Von Frey filaments.

FIG. 3 is a graphical representation of flupirtine dose response curves compared with saline control and GABAPentin 50 mg/kg in reversal of carrageenan-induced allodynia assessed with Von Frey filament: effects of coadministration of aprepitant 3.12 mg/kg.

### DETAILED DESCRIPTION

It is to be understood that unless otherwise indicated, the subject invention is not limited to specific formulations of components, manufacturing methods, dosage regimes, methods of treatment, uses and the like, as such may vary. It is also to be understood that the terminology used herein is for the purpose of describing particular embodiments only, and is not intended to be limiting.

The singular forms “a”, “an” and “the” include plural aspects unless the context clearly dictates otherwise. Thus, for example, reference to “an opioid” includes a single opioid, as well as two or more opioids; reference to “an NK antagonist” includes a single antagonist, as well as two or more antagonists, reference to “the invention” includes one aspect or multiple aspects of the or an invention.

In the present disclosure, the following terminology is used in accordance with the definitions set forth below.

The terms “compound”, “agent”, “active agent”, “chemical agent”, “pharmacologically active agent”, “medicament”, “active” and “drug” are used interchangeably herein to refer to a chemical compound that induces a desired pharmacological and/or physiological effect. The terms also encompass pharmaceutically acceptable and pharmacologically active ingredients of those active agents specifically mentioned herein including but not limited to salts, esters, amides, prodrugs, active metabolites, analogs and the like. When the terms “compound”, “agent”, “active agent”, “chemical agent” “pharmacologically active agent”, “medicament”, “active” and “drug” are used, then it is to be understood that this includes the active agent per se as well as pharmaceutically acceptable, pharmacologically active salts, esters, amides, prodrugs, metabolites, analogs, etc.

Reference to a “compound”, “agent”, “active agent”, “chemical agent” “pharmacologically active agent”, “medicament”, “active” and “drug” includes combinations of two or more actives such as two or more opioids. A “combination” also includes multi-part compositions such as a two-part composition where the agents are provided separately and given or dispensed separately or admixed together prior to dispensation.

For example, a multi-part pharmaceutical pack may have two or more active agents separately maintained. The pharmaceutical pack may also have instructions for use. The instructions may be in the form of a therapeutic protocol.

The terms “effective amount” and “therapeutically effective amount” of an agent as used herein mean a sufficient amount of the agent (e.g. an NK antagonist and/or flupirtine) to provide the desired therapeutic or physiological effect or outcome, which includes achievement of pain reduction such as analgesia in relation to inflammatory pain. Undesirable effects, e.g. side effects (e.g. overt sedation), are sometimes manifested along with the desired therapeutic effect; hence, a practitioner balances the potential benefits against the potential risks in determining what is an appropriate “effective amount”. The exact amount required will vary from subject to subject, depending on the species, age and general condition of the subject, mode of administration and the like. Thus, it may not be possible to specify an exact “effective amount”. However, an appropriate “effective amount” in any individual case may be determined by one of ordinary skill in the art using only routine experimentation. In particular, the methods and compositions described herein including the therapeutic protocol achieve analgesia of inflammatory pain without overt sedation.

By “pharmaceutically acceptable” carrier, excipient or diluent is meant a pharmaceutical vehicle comprised of a material that is not biologically or otherwise undesirable, i.e. the material may be administered to a subject along with the selected active agent without causing any or a substantial adverse reaction. Carriers may include excipients and other additives such as diluents, detergents, coloring agents, wetting or emulsifying agents, pH buffering agents, preservatives, and the like.

Similarly, a “pharmacologically acceptable” salt, ester, emide, prodrug or derivative of a compound as provided herein is a salt, ester, amide, prodrug or derivative that this not biologically or otherwise undesirable.

The terms “treating” and “treatment” as used herein refer to reduction in severity and/or frequency of pain associated with the condition being treated, elimination of symptoms and/or underlying cause of the pain, prevention of the occurrence of pain associated with the condition and/or their underlying cause and improvement or remediation or amelioration of pain following a condition. Hence, the treatment proposed herein reduces pain but this may be independent of the condition being treated.

“Treating” a subject may involve both treating the condition and reducing inflammatory pain.

A “subject” as used herein refers to an animal, including a mammal including a human who can benefit from the pharmaceutical formulations and methods of the present invention. There is no limitation on the type of animal that could benefit from the presently described pharmaceutical formulations and methods. A subject regardless of whether a human or non-human animal may be referred to as an individual, patient, animal, host or recipient. The compounds and methods described herein have applications in human medicine, veterinary medicine as well as in general, domestic or wild animal husbandry.

As indicated above, the methods and compositions are suitable for humans or other primates such as orangutangs, gorillas and marmosets as well as livestock animals, laboratory test animals, companion animals or captive wild animals, and avian species.

Examples of laboratory test animals include mice, rats, rabbits, simian animals, guinea pigs and hamsters. Rabbits, rodent and simian animals provide a convenient test system or animal model. Livestock animals include sheep, cows, pigs, goats, horses and donkeys.

In one aspect, a method is provided for inducing an analgesic response without inducing overt sedation to inflammatory pain in a mammal. In this context the term “mammal” is intended to encompass both humans and other mammals such as laboratory test animals. This aspect also includes, in one embodiment, the step of selecting a subject having inflammatory pain to be a recipient of treatment. The selection process includes an assessment of symptoms of inflammatory pain or symptoms of a condition likely to result in inflammatory pain.

The term “inflammatory pain” is intended to describe the subset of acute and chronic pain that results from inflammatory processes, such as may arise in the case of infections, arthritis and neoplasia or tumor related hypertrophy. Tumor or cancer associated pain is, therefore, considered to fall within the category of inflammatory pain. Examples of conditions associated with inflammatory pain include rheumatoid arthritis, osteo-arthritis, psoriatic arthropathy, arthritis associated with other inflammatory and autoimmune conditions, degenerative conditions such as back strain and mechanical back pain or disc disease, post operative pain, pain from an injury such as a soft tissue bruise or strained ligament or broken bone, abscess or cellulitis, fibrositis or myositis.

In certain methods described herein, an analgesic response is induced without inducing overt sedation to inflammatory pain being suffered by a mammalian subject, including a human subject. A subject, in this context, is also referred to as a “patient”, “target” or “recipient”. In this context the terms “analgesia” and “analgesic response” are intended to describe a state of reduced sensibility to pain, which occurs without overt sedation and in an embodiment without an effect upon the sense of touch. In another aspect, the sensibility to pain is reduced by at least 10%, at least 20%, at least 50%, at least 70% or at least 85% including at least 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84 or 85% . In another aspect, the sensibility to the inflammatory pain is completely, or substantially completely, removed. To assess the level of reduction of sensibility to pain associated with the analgesia induced by the methods according to the present invention it is possible to conduct tests such as the short form McGill pain questionnaire and/or visual analog scales for pain intensity and/or verbal rating scales for pain intensity and/or measurement of tactile allodynia using von Frey hairs or similar device. These tests are standard tests within the art and would be well known to the skilled person.

Accordingly, a method is contemplated for inducing an analgesic response without overt sedation to inflammatory pain in a mammal comprising administering to the subject an amount of an NK antagonist and a neuronal excitation inhibitor or a pharmaceutically acceptable salt, derivative, homolog or analog thereof effective to reduce the level of or otherwise ameliorate the sensation of pain. Examples of particular neuronal excitation inhibitors include flupirtine and retigabine or their pharmaceutically acceptable salts, derivatives, homologs or analogs.

Another aspect provides a method of inducing analgesia without overt sedation in a mammal suffering inflammatory pain by administering to the mammal an NK antagonist concurrently, separately or sequentially with respect to a neuronal excitation inhibitor, such as flupirtine or retigabine, or a pharmaceutically acceptable salt, derivative, homolog or analog thereof, in an amount effective to reduce the level of or otherwise ameliorate the sensation of pain associated with inflammation without inducing overt sedation.

Still another aspect contemplates combination therapy in the treatment of inflammation without inducing overt sedation wherein the treatment of the disease, condition or pathology is conducted in association with pain management using an NK antagonist and a neuronal excitation inhibitor, such as flupirtine or retigabine or a pharmaceutically acceptable salt, derivative, homolog or analog thereof and optionally in addition to an analgesic agent.

Even still another aspect provides a method for inducing an analgesic response to inflammatory pain without inducing overt sedation in a mammal comprising administering to the subject an amount of an NK antagonist and a sodium channel blocker such as but not limited to lamotrogine and mexilentine or a pharmaceutically acceptable salt, derivative, homolog or analog thereof to reduce the level of or otherwise ameliorate the sensation of pain without inducing overt sedation.

Yet another aspect is directed to a method for inducing an analgesic response to inflammatory pain without inducing overt sedation in a mammal comprising administering to the subject an amount of an NK antagonist and a local anaesthetic such as lignocaine, bupivacaine, ropivacaine, and procaine tetracaine or a pharmaceutically acceptable salt, derivative, homolog or analog thereof to reduce the level of or otherwise ameliorate the sensation of pain without inducing overt sedation.

Furthermore, the NK antagonist may be used in combination with one or more modulators of TRPV1 receptors, such as but not limited to capsaicin, capsazepine, Nb-VNA, Nv-VNA, SB-705498 and anadamide or a pharmaceutically acceptable salt, derivative, homolog or analog thereof.

Still further, the NK antagonist may be used in combination with one or more modulators of CB2 receptors such as but not limited to SR144528, AM630 and anadamide or a pharmaceutically acceptable salt, derivative, homolog or analog thereof.

The analgesic effect is without overt sedation.

By the term “overt sedation” it is intended to convey that the methods (and compositions) described herein do not result in a level of sedation of the patient or subject being treated which shows significant, visible or apparent drowsiness or unconsciousness of the patient being treated. Thus, the treatment methods and compositions herein do not result in sleepiness or drowsiness in the patient that interfere with, or inhibit, the activities associated with day to day living, such as driving a motor vehicle or operating machinery for human subjects, or feeding and grooming for animal subjects. Without overt sedation also means inducing an analgesic effect without causing significant cognitive or general impairment of nervous system function (such as attentiveness or wakefulness). Such effects on cognition can lead to a change in the measurement that leads to an erroneous conclusion about the level or type of pain or effect of amelioration of symptoms.

The term “NK antagonist” is intended to encompass known and as yet unknown compounds (including pharmaceutically acceptable salts, derivatives, homologs or analogs thereof) that are effective for treatment of pain in mammals, including compounds which act directly on NK1, NK2 or substance P to inhibit its activity or compounds which act on the family of NK receptors such as NK1, NK2 and NK3 receptors. Examples of such agents include achiral pyridine class of neurokinin-1 receptor antagonists; netupitant 21, betctupitant 29; elzlopitant; lanepitant; osanetant; talnetant; GR205171; MEN 11467; nepadutant; (MEN 11420); M274773; [Sar (9), Met (02) (11)]-Substance P; Tyr (6), D-Phe (7), D-His (9)-Substance-P (6-11) (sendide); (beta;-Ala(8))-Neurokinin A (4-10); (Tyr(5), D-Trp (6,8,9), Lys-NH(2) (10))-Neurokinin A; [D-Proz, D-Trip 7,9]-SP DPDT-SP; [D-Proz, D-Phe7, D-Trp9]-SP; SR48968 and 4-Alkylpiperidine derivative; SB223412; MDL103392; phosphorylated morpholine acetal human neurokinin-1 receptor agonists; SDZ NKT 343; LY 303 870; Ym-35375 and spiro-substituted piperidines; YM-44778; YM-38336; Septide; L732,13:; Dactinomyan analogues; MEN 10207; L 659874; L 668,169; FR113680 and derivative; GR 83074; tripeptides possersi, the glutaminyl-D-trypto phy phenyl alonite sequence; L 659,877; R396; Imidazo[4,5-b]quinoxaline cyonines as neurokinin antagonists; MEN 10208; DPDTP-octa; GR73632; GR64349; senktide; GR71251; [D-Argl, D-Pro2, D-Trp 7,9, Leull]-SP (1-11); Ac heu-Asp-Gln-Trp-Phe-Gly NH2; Thr-Asp-Tyr-D-Tvp-Val-D-Trp-D-Trp-Arg NH2; Cyclo [Eln-Trp-Phe-Gly-Leu-Met]; D-Pro2D-Trp 7,9; D-ArglD-Trp 7,9 leul 1; [Gly6]-NKB [3-10]; [Arg3, D-Ala6]-NKB [3-10]; CP-9634; 3 aminoquinudidine; CP-99994; S18525; S19752; 4-quinoline carboxinide fremincik class; CP-122721; MK-869; GR205171; Spantide II; CP-96,345; L703,606; SR140,333; 2-phenyl-4-quinolinecarboximides class; FK224; FK888; ZM253270-pyrrolopyrimidine class of nonpeptide neurokinin antagonists; GR71251; GR82334; RP67580; diacylpiperazine antagonists of human neurokinin eg L-161664; RP67580; MEN 10376; GR98400; N2-[N2-(1H-indol-3-ylcarbonyl)-L-lysyl]-N-methyl-N-(phenyl-methyl)-L-phenylalaninamibe (2b); SP-(1-11); SP-(6-11); SP-(4-11) WIN51703; Spantide II; Spantide III; Spantide I; aprepitant; MEN13510; 1-[2-(R)-{1-1R)-[3,5-bis (trifluoromethyl)phenyl]ethoxy}-3-(R)-(3,4-difluorophenyl)-4-(R)-tetrahydro-2H-pyran-4-ylmethyl]-3-(r)-methylpiperidine-3-carboxylic acid (1); LY 306,740; SLV-323; 2 substituted-4-aryl-6,7,8,9-tetrahydro-5H-pyrimido [4,5-b][1,5]oxazocin-5-one; 9-substituted-7-aryl-3,4,5,6-tetrahydro-2H-pyrido[4,3-b]- and [2,3-b]-1,5-oxazocin-6-one, SR142801; SB222200; CP96345; SR48968; ezlopitant; MEN11558; [18F] SPA-RQ; neuropitant 21; betupitant 29; SR 144190; SR48692; SR141716; L733060; vofopitant; R-673; nepadutant; saredutant; UK 290795; 2-(4-biphenylyl)quinoline-4-carboxylate and carboxamide analogs (neurokinin-3 receptor antagonist); 4-Amino-2-(aryl)-butylbenzamides and analogues; MK-869; L742694; CP 122721; 1-alkyl-5-(3,4-dichlorophenyl)-5-[2-[(3-substiuted)-1-azetidinyl]ethyl]-2-piperidines; L760735; L758,298, Cbz-Gly-Leu-Trp-OBzl(CF(3))(2); L733,061; SR144190; SB235375; N—[(R,R)-(E)-1-arylmethyl-3-(2-oxo-azepan-3-yl) carbamoyl]allyl-N-methyl-3,5-bis(trifluoromethyl) benzamides; 3-[N′-3,5-bis(trifluoromethyl) benzoyl-N-arylmethyl-N¹-methylhydrazino]-N—[(R)-2-oxo-azepan-3-yl]propionanides; SR142806; SR48,968; CP141,938; LY306740; SB40023; SB414240; SR140333; perhydroisoindole RP 67580, Depitant; RPR 100893; Lanepitant; LY-303870; LY303870; nolpitanium; SR 140333; SR 48968 and Savedutant.

As used herein, compounds which inhibit neuronal excitation include, without being limited to, flupirtine or retigabine; compounds which cause opening of neuronal potassium channels, opioids, neurosteroids, NSAIDS; NMDA receptor antagonists and calcium channel antagonists.

Potassium channels openers contemplated for use in the present invention include, without being limited to flupirtine, Retigabine, WAY-133537, ZD6169, Celikalim, NN414, arycyclopropylcarboxylic amides, 3-(pyridinyl-piperazin-1-YL)-phenylethyl amides, cromakalim, pinacidil, P1060, SDZ PC0400, minoxidil, nicrandil, BMS-204352, cromokalim, leveromakalim, lemakalim, diazoxide, charybdotoxin, glyburide and 4-aminopyridine.

Sodium channel blockers include lamotrogine and mexilentine.

Local anaesthetics include lignocaine, bupivacaine, ropivacaine, procaine and tetracaine.

Reference to a “neuronal excitation inhibitor” may also include a sodium channel blocker, a local anaesthetic, a modulator of TRPV1 receptor and/or modulator of CB2 receptor. Equally, a sodium channel blocker, a local anaesthetic, a modulator of TRPV1 receptor and/or modulator of CB2 receptor may also be a neuronal excitation inhibitor.

A modulator of TRPV1 receptor includes but is not limited to capsaicin, capsazepine, Nb-VNA, Nv-VNA, SB-705498 and anadamide or a pharmaceutically acceptable salt, derivative, homolog or analog thereof.

The modulator may be an agonist or an antagonist of the TRPV1 receptor. SB-705498 is an example of an antagonist and capsaicin, capsazepine, Nb-VNA, Nv-VNA and anadamide are examples of agonists.

A modulator of CB2 receptor includes but is not limited to SR144528, AM630 and anandamide or a pharmaceutically acceptable salt, derivative, homolog or analog thereof. The modulator may be an agonist or an antagonist of the CB2 receptor.

As used herein, opioid compounds (opioids) include any compound that is physiologically acceptable in mammalian systems and is a full or at least partial agonist of an opioid receptor, Opioid compounds are well known and include naturally occurring compounds derived from opium such as codeine, morphine and papavarine as well as derivatives of such compounds that generally have structural similarity as well as other structurally unrelated compounds that agonise an opioid receptor present in a mammalian system. Specific examples of opioid compounds contemplated by the present invention include: fentanyl, oxycodone, codeine, dihydrocodeine, dihydrocodeinone enol acetate, morphine, desomorphine, apomorphine, diamorphine, pethidine, methadone, dextropropoxyphene, pentazocine, dextromoramide, oxymorphone, hydromorphone, dihydromorphine, noscapine, nalbuprhine papaverine, papavereturn, alfentanil, buprenorphine and tramadol and pharmaceutically acceptable salts, derivatives, homologs or analogs thereof.

Neurosteroids contemplated for use in the present invention include alphadolone and other pregnanediones and salts and derivates thereof (eg alphadolone mono and bi glucuronides) and other neurosteroids that cause antinociception without overt sedation by interaction with spinal cord GABAa receptors.

As used herein, an NMDA receptor antagonist is an agent which blocks or inhibits the activity and/or function of NMDA receptors. Hence, the present invention extends to functional NMDA antagonists as well as structural NMDA antagonists. The NMDA receptor is a cell-surface protein complex, widely distributed in the mammalian central nervous system that belongs to the class of ionotropic-glutamate receptors. It is involved in excitatory-synaptic transmission and the regulation of neuronal growth. The structure comprises a ligand-gated/voltage-sensitive ion channel. The NMDA receptor is highly complex and is believed to contain at least five distinct binding (activation) sites: a glycine-binding site, a glutamate-binding site (NMDA-binding site); a PCP-binding site, a polyamine-binding site, and a zinc-binding site. In general, a receptor antagonist is a molecule that blocks or reduces the ability of an agonist to activate the receptor. As used herein, an “NMDA-receptor antagonist” means any compound or composition, known or to be discovered, that when contacted with an NMDA receptor in vivo or in vitro, inhibits the flow of ions through the NMDA-receptor ion channel. A “functional” NMDA antagonist includes agents which raise the threshold for NMDA receptor activation Activating NMDA receptors increases cell excitability. Any drug that inhibits or decreases neuronal excitation in the CNS can potentially be a “functional” NMDA receptor antagonist because it decreases the excitation caused by NMDA receptor agonists. All such agents may be used in combination with NK antagonists to achieve a desired analgesic effect.

An NMDA-receptor antagonist can contain one or more chiral centers and/or double bonds and, therefore, exist as stereoisomers, such as double-bond isomers (i.e., geometric isomers), enantiomers, or diastereomers. As used herein, the term “NMDA-receptor antagonist” encompass all such enantiomers and stereoisomers, that is, both the stereomerically-pure form (e.g., geometrically pure, enantiomerically pure, or diastereomerically pure) and enantiomeric and stereoisomeric mixtures, e.g., racemates. The term “NMDA-receptor antagonist” further encompasses all pharmaceutically acceptable salts, all complexes (e.g., hydrates, solvates, and clathrates), and all prodrugs of NMDA-receptor antagonist.

NMDA-receptor antagonists suitable for use in the invention can be identified by testing NMDA-receptor antagonists for antinociceptive properties according to standard pain models. See e.g., Sawynok et al. Pain 82:149, 1999; Sawynok et al. Pain 80:45, 1999.

In an aspect, the NMDA-receptor antagonist is a non-competitive NMDA-receptor antagonists, more preferably, ketamine, even more preferably, ketamine hydrochloride.

As used herein the meaning of the phrase “NMDA-receptor antagonist” encompasses any compound or composition that antagonizes the NMDA receptor by binding at the glycine site. For a review on glycine-site NMDA-receptor antagonists, see Leeson, P. D. Drug Design for Neuroscience 13:338-381, 1993. Glycine-site NMDA-receptor antagonists can be identified by standard in vitro and in vivo assays. See, for example, the assays described in U.S. Pat. No. 6,251,903 (issued Jun. 26, 2001); U.S. Pat. No. 6,191,165 (issued Feb. 20, 2001; Grimwood et al. Molecular Pharmacology 4:923 1992; Yoneda et al. J Neurochem 62:102, 1994; and Mayer et al. J Neurophysiol 645, 1988, all of which citations are hereby expressly incorporated herein by reference.

Glycine-site NMDA-receptor antagonists include, but are not limited to, glycinamide, threonine, D-serine, felbamate, 5,7-dichlorokynurenic acid, and 3-amino-1-hydroxy-2-pyrrolidone (HA-966), diethylenetriamine, 1,10-diaminodecane, 1,12-diaminododecane, and ifenprodil and those described in U.S. Pat. Nos. 6,251,903; 5,914,403 (issued Jun. 22, 1999); U.S. Pat. No. 5,863,916 (issued Jan. 26, 1999); U.S. Pat. No. 5,783,700 (issued Jul. 21, 1998); and U.S. Pat. No. 5,708,168 (issued Jan. 13, 1998), all of which patents are hereby expressly incorporated herein by reference.

As used herein the meaning of the phrase “NMDA-receptor antagonist” encompasses any compound or composition that antagonizes the NMDA receptor by binding at the glutamate site also referred to herein as “competitive NMDA-receptor antagonists”; see, for example, Olney & Farber Neuropsychopharmacology 13:335, 1995.

Competitive NMDA antagonists include, but are not limited to, 3-((−)-2-carboxypiperazin-4-ylpropyl-1-phosphate (CPP); 3-(2-carboxypiperzin-4-yl)-prpenyl-1-phosphonate (CPP-ene); 1-(cis-2-carboxypiperidine-4-yl)methyl-1-phosphonic acid (CGS 19755), D-2-Amino-5-phosphonopentanoic acid (AP5); 2-amino-phosphonoheptanoate (AP7); D,L-(E)-2-amino-4-methyl-5-phosphono-3-pentenoic acid carboxyethyl ester (CGP39551); 2-amino-4-methyl-5-phosphono-pent-3-enoic acid (CGP 40116); (4-phosphono-but-2-enylamino)-acetic acid (PD 132477); 2-amino-4-oxo-5-phosphono-pentanoic acid (MDL 100,453); 3-((phosphonylmethyl)-sulfinyl)-D,L-alanine; amino-(4-phosphonomethyl-phenyl)-acetic acid (PD 129635); 2-amino-3-(5-chloro-1-phosphonomethyl-1H-benzoimidazol-2-yl)-propionic acid; 2-amino-3-(3-phosphonomethyl-quinoxalin-2-yl)-propionic acid; 2-amino-3-(5-phosphonomethyl-biphenyl-3-yl)-propionic acid (SDZ EAB 515); 2-amino-3-[2-(2-phosphono-ethyl)-cyclohexyl]-propionic acid (NPC 17742); 4-(3-phosphono-propyl)-piperazine-2-carboxylic acid (D-CPP); 4-(3-phosphono-allyl)-piperazine-2-carboxylic acid (D-CPP-ene); 4-phosphonomethyl-piperidine-2-carboxylic acid (CGS 19755); 3-(2-phosphono-acetyl)-piperidine-2-carboxylic acid (MDL 100,925); 5-phosphono-1,2,3,4-tetrahydro-isoquinoline-3-carboxylic acid (SC 48981); 5-(2-phosphono-ethyl)-1,2,3,4-tetrahydro-isoquinoline-3-carboxylic acid (PD 145950); 6-phosphonomethyl-decahydro-isoquinoline-3-carboxylic acid (LY 274614); 4-(1H-tetrazol-5-ylmethyl)-piperidine-2-carboxylic acid (LY 233053 and 235723); and 6-(1H-Tetrazol-5-ylmethyl)-decahydro-isoquinoline-3-carboxylic acid (LY 233536).

As used herein the meaning of the phrase “NMDA-receptor antagonist” encompasses any compound or composition that antagonizes the NMDA receptor by binding at the PCP (phencyclidine) site, referred to herein as “non-competitive NMDA-receptor antagonists”.

Non-competitive NMDA-receptor antagonists can be identified using routine assays, for example, those described in U.S. Pat. No. 6,251,948 (issued Jun. 26, 2001); U.S. Pat. No. 5,985,586 (issued Nov. 16, 1999), and U.S. Pat. No. 6,025,369 (issued Feb. 15, 2000); Jacobson et al J Pharmacol Exp Ther 110.243, 1987; and Thurkauf et al J Med Chem 31:2257, 1988, all of which citations are hereby expressly incorporated herein by reference.

Examples of non-competitive NMDA-receptor antagonists that bind at the PCP site include, but are not limited to, ketamine, phencyclidine, dextromethorphan, dextrorphan, dexoxadrol, dizocilpine (MK-801), remacemide, thienylcyclohexylpiperidine (TCP), N-allylnometazocine (SKF 10,047), cyclazocine, etoxadrol, (1,2,3,4,9,9a-hexahydro-fluoren-4a-yl)-methyl-amine (PD 137889); (1,3,4,9,10,10a-hexahydro-2H-phenanthren-4a-yl)-methyl-amine (PD 138289); PD 138558, tiletamine, kynurenic acid, 7-chloro-kynurenic acid, and memantine; and quinoxalinediones, such as 6-cyano-7-nitroquinoxaline-2,3-dione (CNQX) and 6,7-dinitro-quinoxaline-2,3-dione (DNQX).

As used herein the meaning of “NMDA-receptor antagonist” encompasses compounds that block the NMDA receptor at the polyamine binding site, the zinc-binding site, and other NMDA-receptor antagonists that are either not classified herein according to a particular binding site or that block the NMDA receptor by another mechanism. Examples of NMDA-receptor antagonists that bind at the polyamine site include, but are not limited to, spermine, spermidine, putrescine, and arcaine. Examples of assays useful to identify NMDA-receptor antagonists that act at the zinc or polyamine binding site are disclosed in U.S. Pat. No. 5,834,465 (issued Nov. 10, 1998), hereby expressly incorporated by reference herein.

Other NMDA-receptor antagonists include, but are not limited to, amantadine, eliprodil, lamotrigine, riluzole, aptiganel, flupirtine, celfotel, levemopamil, 1-(4-hydroxy-phenyl)-2-(4-phenylsulfanyl-piperidin-1-yl)-propan-1-one; 2-[4-(4-fluoro-benzoyl)-piperidin-1-yl]-1-naphthalen-2-yl-ethanone (E 2001); 3-(1,1-dimethyl-heptyl)-9-hydroxymethyl-6,6-dimethyl-6a,7,8,10a-tetrahydro -6H-benzo[c]chromen-1-ol (HU-211); 1-{4-[1-(4-chloro-phenyl)-1-methyl-ethyl]-2-methoxy-phenyl}-1H-[1,2,4]tria zole-3-carboxylic acid amide (CGP 31358); acetic acid 10-hydroxy-7,9,7′,9′-tetramethoxy-3,3′-dimethyl-3,4,3′,4′-tetrahydro-1H,1′H-[5,5′]bi[benzo[g]isochromenyl]-4-yl ester (ES 242-1); 14-hydroxy-11 isopropyl-10-methyl-5-octyl-10,13-diaza-tricyclo[6.6.1.04,15]pentadeca-1,4,6,8(15)-tetraen-12-one; and 4,5-dioxo-4,5-dihydro-1H-benzo[g]indole-2,7,9-tricarboxylic acid (PQQ) and pharmaceutically acceptable salts thereof.

Calcium channel antagonists include diltiazem, ziconotide (MVIIA), CVID (AM336), NMED-160, cilnidipine, GABApentin and pregabalin.

NSAIDS include, without being limited to, NSAIDS, such as acetaminophen (Tylenol, Datril, etc.), aspirin, ibuprofen (Motrin, Advil, Rufen, others), choline magnesium salicylate (Triasate), choline salicylate (Anthropan), diclofenac (voltaren, cataflam), diflunisal (dolobid), etodolac (Iodine), fenoprofen calcium (nalfon), fluorobiprofen (ansaid), indomethacin (indocin, indometh, others), ketoprofen (orudis, oruvail), ketorolac tromethamine (toradol), magnesium salicylate (Doan's, magan, mobidin, others), meclofenamate sodium (meclomen), mefenamic acid (relafan), oxaprozin (daypro), piroxicam (feldene), sodium salicylate, sulindac (clinoril), tolmetin (tolectin), meloxicam, nabumetone, naproxen, lornoxicam, nimesulide, indoprofen, remifenzone, salsalate, tiaprofenic acid, flosulide, and the like.

The phrase “pharmaceutically acceptable salt, derivative, homologs or analogs” is intended to convey any pharmaceutically acceptable tautomer, salt, pro-drug, hydrate, solvate, metabolite or other compound which, upon administration to the subject, is capable of providing (directly or indirectly) the compound concerned or a physiologically (e.g. analgesically) active compound, metabolite or residue thereof. An example of a suitable derivative is an ester formed from reaction of an OH or SH group with a suitable carboxylic acid, for example C₁₋₃alkyl-CO₂H, and HO₂C—(CH₂)ₙ—CO₂H (where n is 1-10 such as 1, 2, 3, 4, 5, 6, 7, 8, 9, 10, but preferably 1-4), and CO₂H—CH₂phenyl.

Thus, the active compounds may be in crystalline form, either as the free compounds or as solvates (e.g. hydrates). Methods of solvation are generally known within the art.

The salts of the active compounds of the invention are preferably pharmaceutically acceptable, but it will be appreciated that non-pharmaceutically acceptable salts also fall within the scope of the present invention, since these are useful as intermediates in the preparation of pharmaceutically acceptable salts. Examples of pharmaceutically acceptable salts include salts of pharmaceutically acceptable cations such as sodium, potassium, lithium, calcium, magnesium, ammonium and alkylammonium; acid addition salts of pharmaceutically acceptable inorganic acids such as hydrochloric, orthophosphoric, sulfuric, phosphoric, nitric, carbonic, boric, sulfamic and hydrobromic acids; or salts of pharmaceutically acceptable organic acids such as acetic, propionic, butyric, tartaric, maleic, hydroxymaleic, fumaric, citric, lactic, mucic, gluconic, benzoic, succinic, oxalic, phenylacetic, methanesulphonic, trihalomethanesulfphonic, toluenesulphonic, benzenesulphonic, salicyclic, sulphanilic, aspartic, glutamic, edetic, stearic, palmitic, oleic, lauric, pantothenic, tannic, ascorbic and valeric acids.

The term “pro-drug” is used herein in its broadest sense to include those compounds which can be converted in vivo to the compound of interest (e.g. by enzymatic or hydrolytic cleavage). Examples thereof include esters, such as acetates of hydroxy or thio groups, as well as phosphates and sulphonates. Processes for acylating hydroxy or thio groups are known in the art, e.g. by reacting an alcohol (hydroxy group), or thio group, with a carboxylic acid. Other examples of suitable pro-drugs are described in Bundgaard Design of Prodrugs, Elsevier 1985, the disclosure of which is included herein in its entirety by way of reference.

The term “metabolite” includes any compound into which the active agents can be converted in vivo once administered to the subject. Examples of such metabolites are glucuronides, sulphates and hydroxylates.

It will be understood that active agents as described herein may exist in tautomeric forms. The term “tautomer” is used herein in its broadest sense to include compounds capable of existing in a state of equilibrium between two isomeric forms. Such compounds may differ in the bond connecting two atoms or groups and the position of these atoms or groups in the compound. A specific example is keto-enol tautomerism.

The compounds of the present invention may be electrically neutral or may take the form of polycations, having associated anions for electrical neutrality. Suitable associated anions include sulfate, tartrate, citrate, chloride, nitrate, nitrite, phosphate, perchlorate, halosulfonate or trihalomethylsulfonate.

The active agents may be administered for therapy by any suitable route. It will be understood that the active agents are preferably administered via a route that does not result in overt sedation of the subject. Suitable routes of administration may include oral, rectal, nasal, inhalation of aerosols or particulates, topical (including buccal and sublingual), transdermal, vaginal, intravesical and parenteral (including subcutaneous, intramuscular, intravenous, intrasternal, intra-articular, injections into the joint, intrathecal, epidural and intradermal). In one embodiment, administration of the active agent is by a route resulting in first presentation of the compound to the stomach of the subject. In this embodiment, the active agents are generally administered via an oral route. In another embodiment the active agents are administered by the transdermal route, However it will be appreciated that the preferred route will vary with the condition and age of the subject, the nature of the inflammatory pain being treated, its location within the subject and the judgement of the physician or veterinarian. It will also be understood that individual active agents may be administered by the same or different distinct routes. The individual active agents may be administered separately or together directly into a joint involved with an inflammatory painful process.

As used herein, an “effective amount” refers to an amount of active agent that provides the desired analgesic activity when administered according to a suitable dosing regime. The amount of active agent is generally an amount that provides the desired analgesic activity without causing overt sedation. Dosing may occur at intervals of several minutes, hours, days, weeks or months. Suitable dosage amounts and regimes can be determined by the attending physician or veterinarian. For example, flupirtine or pharmaceutically acceptable salts, derivatives, homologs or analogs thereof, may be administered to a subject at a rate of between about 0.5 to about 20 mg/kg by body weight every from about 1 hour to up to about 50 hours, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 hours in amounts of 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5 or 20 mg/kg. Particularly useful times are from about 6 hours to about 24 hours, such as 6, 7, 8, 9, 10, 11, 12, 13, 14, 15, 16, 17, 18, 19, 20, 21, 22, 23, 24. Even more particular useful times are between from about 12 to about 24 hours. Such as 12, 13, 14, 15, 16, 17, 18, 19, 20, 21, 22, 23 or 24 hours. Dosing of the analgesic agent, such as an opioid, can be determined by the attending physician in accordance with dosing rates in practice. For example, fentanyl can be administered in an amount of about 100 μg whereas morphine may be administered in an amount of 10 mg, also on an hourly basis. The administration amounts may be varied if administration is conducted more or less frequently, such as by continuous infusion, by regular dose every few minutes (e.g. 1, 2, 3 or 4 minutes) or by administration every 5, 10, 20, 30 or 40 minutes (e.g. 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 45, 36, 37, 38, 39 or 40 minutes) or every 1, 2, 3, 4, 5, 6, 7, 8, 9, 10, 11, 12, 13, 14, 15, 16, 17, 18, 19, 20, 21, 22, 23 or 24 hours or up to 50 hours such as, for example, 25, 26, 27, 28, 29, 30, 31, 32, 33, 34, 35, 36, 37, 38, 39, 40, 41, 42, 43, 44, 45, 46, 47, 48, 49 or 50 hours. In many instances administration will be conducted simply on the basis of when the patient requires pain relief.

In one particular embodiment, one or more NK antagonist in combination with the neuronal excitation inhibitor(s) is used to treat inflammatory pain associated with inflammatory diseases or conditions.

“Inflammatory diseases and disorders” encompass those disease and disorders which result in a response of redness, swelling, pain, and a feeling of heat in certain areas that is meant to protect tissues affected by injury or disease. Pain associated with the following inflammatory diseases can be treated using the methods of the present invention: acne, angina, arthritis, aspiration pneumonia, disease, empyema, gastroenteritis, inflammation, intestinal flu, necrotizing enterocolitis (NEC), pelvic inflammatory disease (PID), pharyngitis, pleurisy, raw throat, redness, rubor, sore throat, stomach flu and urinary tract infections, Chronic Inflammatory Demyelinating Polyneuropathy, post-operative pain and Chronic Inflammatory Demyelinating Polyradiculoneuropathy.

Accordingly, a treatment protocol is contemplated for treating inflammatory pain without inducing overt sedation in a subject, the protocol comprising the steps of administering to the subject an effective amount of an anti-inflammatory agent in conjunction with one or more NK antagonist and an inhibitor of neuronal excitation. The inflammatory disease may include any of those listed above. Administration of the anti-inflammatory agent may be sequential or simultaneous or independent of the neuronal excitation inhibitor and the NK antagonist.

Another aspect also provides a composition comprising one or more NK antagonist or a pharmaceutically acceptable salt, derivative, homolog or analog thereof, with an inhibitor of neuronal excitation together with one or more pharmaceutically acceptable additives and optionally other medicaments. The pharmaceutically acceptable additives may be in the form of carriers, diluents, adjuvants and/or excipients and they include all conventional solvents, dispersion agents, fillers, solid carriers, coating agents, antifungal or antibacterial agents, dermal penetration agents, surfactants, isotonic and absorption agents and slow or controlled release matrices. The active agents may be presented in the form of a kit of components adapted for allowing concurrent, separate or sequential administration of the active agents. Each carrier, diluent, adjuvant and/or excipient must be “pharmaceutically acceptable” in the sense of being compatible with the other ingredients of the composition and physiologically tolerated by the subject. The compositions may conveniently be presented in unit dosage form and may be prepared by methods well known in the art of pharmacy. Such methods include the step of bringing into association the active ingredient with the carrier, which constitutes one or more accessory ingredients. In general, the compositions are prepared by uniformly and intimately bringing into association the active ingredient with liquid carriers, diluents, adjuvants and/or excipients or finely divided solid carriers or both, and then if necessary shaping the product.

Compositions of the present invention suitable for oral administration may be presented as discrete units such as capsules, sachets or tablets each containing a predetermined amount of the active ingredient; as a powder or granules; as a solution or a suspension in an aqueous phase or non-aqueous liquid; or as an oil-in-water liquid emulsion or a water-in-oil emulsion. The active ingredient may also be presented as a bolus, electuary or paste.

A tablet may be made by compression or moulding, optionally with one or more accessory ingredients. Compressed tablets may be prepared by compressing in a suitable machine the active ingredient in a free-flowing form such as a powder or granules, optionally mixed with a binder (e.g. inert diluent, preservative disintegrant, sodium starch glycollate, cross-linked povidone, cross-linked sodium carboxymethyl cellulose) surface-active or dispersing agent. Moulded tablets may be made my moulding in a suitable machine a mixture of the powdered compound moistened with an inert liquid diluent. The tablets may optionally be coated or scored and may be formulated so as to provide slow or controlled release of the active ingredient therein using, for example, hydroxypropylmethyl cellulose in varying proportions to provide the desired release profile. Tablets may optionally be provided with an enteric coating, to provide release in parts of the gut other than the stomach.

Compositions suitable for parenteral administration include aqueous and non-aqueous isotonic sterile injection solutions which may contain anti-oxidants, buffers, bacteriostats and solutes which render the composition isotonic with the blood of the intended subject; and aqueous and non-aqueous sterile suspensions which may include suspended agents and thickening agents. The compositions may be presented in a unit-dose or multi-dose sealed containers, for example, ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets of the kind previously described. When reconstituted these can be in the form of aqueous solution, dissolved in water, isotonic saline or a balanced salt solution. Additionally, when reconstituted the product could be a suspension in which the compound(s) is/are dispersed in the liquid medium by combination with liposomes or a lipid emulsion such as soya bean.

Compositions suitable for topical administration to the skin, i.e. transdermal administration, may comprise the active agents dissolved or suspended in any suitable carrier or base and may be in the form of lotions, gels, creams, pastes, ointments and the like. Suitable carriers may include mineral oil, propylene glycol, waxes, polyoxyethylene and long chain alcohols. Transdermal devices, such as patches may also be used and may comprise a microporous membrane made from suitable material such as cellulose nitrate/acetate, propylene and polycarbonates. The patches may also contain suitable skin adhesive and backing materials.

The active compounds described herein may also be presented as implants, which may comprise a drug bearing polymeric device wherein the polymer is biocompatible and non-toxic. Suitable polymers may include hydrogels, silicones, polyethylenes and biodegradable polymers.

The compounds of the subject invention may be administered in a sustained (i.e. controlled) or slow release form. A sustained release preparation is one in which the active ingredient is slowly released within the body of the subject once administered and maintains the desired drug concentration over a minimum period of time. The preparation of sustained release formulations is well understood by persons skilled in the art. Dosage forms may include oral forms, implants and transdermal forms, joint injections, sustained or slow release injectables. For slow release administration, the active ingredients may be suspended as slow release particles or within liposomes, for example.

The compositions herein may be packaged for sale with other active agents or alternatively, other active agents may be formulated with flupirtine or its pharmaceutical salts, derivatives, homologs or analogs thereof and optionally an analgesic agent such as an opioid. The composition may be sold or provided with a set of instructions in the form of a therapeutic protocol. This protocol may also include, in one embodiment, a selection process for type of patient or type of condition or a type of pain.

Thus, a further aspect provides a system for the controlled release of active compounds selected from an NK antagonist in combination with a neuronal excitation inhibitor or a pharmaceutically acceptable salt, derivative, homolog or analog thereof, alone or together with another analgesic or active agent, wherein the system comprises:
(a) a deposit-core comprising an effective amount of a first active compound and having defined geometric form, and
(b) a support-platform applied to the deposit-core, wherein the support-platform contains a second active compound, and at least one compound selected from the group consisting of:
   (i) a polymeric material which swells on contact with water or aqueous liquids and a gellable polymeric material wherein the ratio of the swellable polymeric material to the gellable polymeric material is in the range 1:9 to 9:1, and
   (ii) a single polymeric material having both swelling and gelling properties, and wherein the support-platform is an elastic support applied to the deposit-core so that it partially covers the surface of the deposit-core and follows changes due to hydration of the deposit-core and is slowly soluble and/or slowly gellable in aqueous fluids.

As used herein, the first active substance is one of (i) an NK antagonist or (ii) a neuronal excitation inhibitor. The second active substance may be (i) or (ii) above.

In another aspect, a system is provided for the controlled release for an NK antagonist and a neuronal excitation inhibitor, wherein the system comprises:
(a) a deposit-core comprising an effective amount of (1) one or more NK antagonists and (2) a neuronal excitation inhibitor, the deposit-core having a defined geometric form; and
(b) a support platform applied to the deposit-core, the support platform comprising at least one compound selected from the group consisting of:
   (i) a polymeric material which swells on contact with water or aqueous liquids and a gellable polymeric material wherein the ratio of the swellable polymeric material to the gellable polymeric material is in the range 1:9 to 9:1, and
   (ii) a single polymeric material having both swelling and gelling properties, and wherein the support-platform is an elastic support applied to the deposit-core so that it partially covers the surface of the deposit-core and follows changes due to hydration of the deposit-core and is slowly soluble and/or slowly gellable in aqueous fluids.

A further aspect contemplates a system for the controlled release for an NK antagonist and a sodium channel blocker, wherein the system comprises:
(a) a deposit-core comprising an effective amount of (1) one or more NK antagonists and (2) a sodium channel blocker, the deposit-core having a defined geometric form; and
(b) a support platform applied to the deposit-core, the support platform comprising at least one compound selected from the group consisting of:
   (i) a polymeric material which swells on contact with water or aqueous liquids and a gellable polymeric material wherein the ratio of the swellable polymeric material to the gellable polymeric material is in the range 1:9 to 9:1, and
   (ii) a single polymeric material having both swelling and gelling properties, and wherein the support-platform is an elastic support applied to the deposit-core so that it partially covers the surface of the deposit-core and follows changes due to hydration of the deposit-core and is slowly soluble and/or slowly gellable in aqueous fluids.

Still a further aspect provides a system for the controlled release for an NK antagonist and a local anaesthetic, wherein the system comprises:
(a) a deposit-core comprising an effective amount of (1) one or more NK antagonists and (2) a local anaesthetic, the deposit-core having a defined geometric form; and
(b) a support platform applied to the deposit-core, the support platform comprising at least one compound selected from the group consisting of:
   (i) a polymeric material which swells on contact with water or aqueous liquids and a gellable polymeric material wherein the ratio of the swellable polymeric material to the gellable polymeric material is in the range 1:9 to 9:1, and
   (ii) a single polymeric material having both swelling and gelling properties, and wherein the support-platform is an elastic support applied to the deposit-core so that it partially covers the surface of the deposit-core and follows changes due to hydration of the deposit-core and is slowly soluble and/or slowly gellable in aqueous fluids.

Even yet a further aspect contemplates a system for the controlled release for an NK antagonist and a modulator of TRPV1 receptor, wherein the system comprises:
(a) a deposit-core comprising an effective amount of (1) one or more NK antagonists and (2) a modulator of TRPV1 receptor, the deposit-core having a defined geometric form; and
(b) a support platform applied to the deposit-core, the support platform comprising at least one compound selected from the group consisting of:
   (i) a polymeric material which swells on contact with water or aqueous liquids and a gellable polymeric material wherein the ratio of the swellable polymeric material to the gellable polymeric material is in the range 1:9 to 9:1, and
   (ii) a single polymeric material having both swelling and gelling properties, and wherein the support-platform is an elastic support applied to the deposit-core so that it partially covers the surface of the deposit-core and follows changes due to hydration of the deposit-core and is slowly soluble and/or slowly gellable in aqueous fluids.

Another aspect provides a system for the controlled release for an NK antagonist and a modulator of CB2 receptor, wherein the system comprises:
(a) a deposit-core comprising an effective amount of (1) one or more NK antagonists and (2) a modulator of CB2 receptor, the deposit-core having a defined geometric form; and
(b) a support platform applied to the deposit-core, the support platform comprising at least one compound selected from the group consisting of:
   (i) a polymeric material which swells on contact with water or aqueous liquids and a gellable polymeric material wherein the ratio of the swellable polymeric material to the gellable polymeric material is in the range 1:9 to 9:1, and
   (ii) a single polymeric material having both swelling and gelling properties, and wherein the support-platform is an elastic support applied to the deposit-core so that it partially covers the surface of the deposit-core and follows changes due to hydration of the deposit-core and is slowly soluble and/or slowly gellable in aqueous fluids.

The support-platform may comprise polymers such as hydroxypropylmethylcellulose, plasticizers such as a glyceride, binders such as polyvinylpyrrolidone, hydrophilic agents such as lactose and silica, and/or hydrophobic agents such as magnesium stearate and glycerides. The polymer(s) typically make up 30 to 90% by weight of the support-platform, for example about 35 to 40%. Plasticizer may make up at least 2% by weight of the support platform, for example about 15 to 20%. Binder(s), hydrophilic agent(s) and hydrophobic agent(s) typically total up to about 50% by weight of the support platform, for example about 40 to 50%.

The tablet coating may contain one or more water insoluble or poorly soluble hydrophobic excipients. Such excipients may be selected from any of the known hydrophobic cellulosic derivatives and polymers including alkylcellulose, e.g. ethylcellulose, hydroxypropyl cellulose, hydroxypropylmethyl cellulose, carboxymethyl cellulose, and derivatives thereof; polymethacrylic polymers, polyvinyl acetate and cellulose acetate polymers; fatty acids or their esters or salts; long chain fatty alcohols; polyoxyethylene alkyl ethers; polyoxyethylene stearates; sugar esters; lauroyl macrogol-32 glyceryl, stearoyl macrogol-32 glyceryl, and the like. Hydroxypropylmethyl cellulose materials are preferably selected from those low Mw and low viscosity materials such as E-Type methocel, and 29-10 types as defined in the USP.

Other agents or excipients that provide hydrophobic quality to coatings may be selected from any waxy substance known for use as tablet excipients. Preferably they have a HLB value of less than 5, and more preferably about 2. Suitable hydrophobic agents include waxy substances such as carnauba wax, paraffin, microcrystalline wax, beeswax, cetyl ester wax and the like; or non-fatty hydrophobic substances such as calcium phosphate salts, e.g. dibasic calcium phosphate.

The coating may contain a calcium phosphate salt, glyceryl behenate, and polyvinyl pyrollidone, or mixtures thereof, and one or more adjuvants, diluents, lubricants or fillers.

Components in the coating may be as follows, with generally suitable percentage amounts expressed as percentage weight of the coating.

Polyvinyl pyrollidone (Povidone) is preferably present in amounts of about 1 to 25% by weight or the coating, more particularly 4 to 12%, e.g. 6 to 8%.

Glyceryl behenate is an ester of glycerol and behenic acid (a C22 fatty acid). Glyceryl behenate may be present as its mono-, di-, or tri-ester form, or a mixture thereof. Preferably it has an HLB value of less than 5, more preferably approximately 2. It may be present in amounts of about 5 to 85% by weight of the coating, more particularly from 10 to 70% by weight, and in certain preferred embodiments from 30 to 50%.

Calcium phosphate salt may be the dibasic calcium phosphate dihydrate and may be present in an amount of about 10 to 90% by weight of the coating, preferably 20 to 80%, e.g. 40 to 75%.

The coating may contain other common tablet excipients such as lubricants, colourants, binders, diluents, glidants and taste-masking agents or flavourants.

Examples of excipients include colourants such a ferric oxide, e.g. yellow ferric oxide; lubricants such as magnesium stearate; and glidants such as silicon dioxide, e.g. colloidal silicon dioxide. Yellow ferric oxide may be used in amounts of about 0.01 to 0.5% by weight based on the coating; magnesium stearate may be present in amounts of 1 to 20% by weight of the coating, more preferably 2 to 10%, e.g. 0.5 to 1.0%; and colloidal silica may be used in amounts of 0.1 to 20% by weight of the coating, preferably 1 to 10%, more preferably 0.25 to 1.0%.

The core comprises in addition to a drug substance, a disintegrating agent or mixtures of disintegrating agents used in immediate release formulations and well know to persons skilled in the art. The disintegrating agents useful in the exercise of the present invention may be materials that effervesce and or swell in the presence of aqueous media thereby to provide a force necessary to mechanically disrupt the coating material.

A core may contain, in addition to the drug substance, cross-linked polyvinyl pyrollidone and croscarmellose sodium.

The following is a list of contemplated core materials. The amounts are expressed in terms of percentage by weight based on the weight of the core.

Cross-linked polyvinyl pyrollidone is described above and is useful as a disintegrating agent, and may be employed in the core in the amounts disclosed in relation to the core.

Croscarmellose sodium is an internally cross-linked sodium carboxymethyl cellulose (also known as Ac-Di-Sol) useful as a disintegrating agent.

Disintegrating agents may be used in amounts of 5 to 30% by weight based on the core. However, higher amounts of certain disintegrants can swell to form matrices that may modulate the release of the drug substance. Accordingly, particularly when rapid release is required after the lag time it is preferred that the disintegrants is employed in amounts of up to 10% by weight, e.g. about 5 to 10% by weight.

The core may additionally comprise common tablet excipients such as those described above in relation to the coating material. Suitable excipients include lubricants, diluents and fillers, including but not limited to lactose (for example the mono-hydrate), ferric oxide, magnesium stearates and colloidal silica.

Lactose monohydrate is a disaccharide consisting of one glucose and one galactose moiety. It may act as a filler or diluent in the tablets of the present invention. It may be present in a range of about 10 to 90%, preferably from 20 to 80%, and in certain preferred embodiments from 65 to 70%.

The core should be correctly located within the coating to ensure that a tablet has the appropriate coating thickness.

In this way, lag times are reliable and reproducible, and intra-subject and inter-subject variance in bioavailability is avoided. It is advantageous to have a robust control mechanism to ensure that tablets in a batch contain cores having the appropriate geometry in relation to the coating. Controls can be laborious in that they require an operator to remove random samples from a batch and to cut them open to physically inspect the quality of the core (i.e. whether it is intact, and whether it is correctly located). Furthermore, if a significant number of tablets from the sample fail, a complete batch of tablets may be wasted. Applicant has found that if one adds to the core a strong colourant such as iron oxide, such that the core visibly contrasts with the coating when as strong light is shone on the tablet, it is possible for any faults in the position or integrity of the core to be picked up automatically by a camera appropriately located adjacent a tabletting machine to inspect tablets as they are ejected therefrom.

Still another aspect provides a composition comprising: (a) one or more NK antagonists; and (b) an immediate release neuronal excitation inhibitor.

A method for the delivery of the composition to a subject is provided comprising the step of administering the composition to the subject orally, transdermally, or subdermally, wherein the composition comprises components (a) and (b) as defined above.

In one aspect, a tamper-proof narcotic delivery system is produced which provides for full delivery of narcotic medication and for analgesic action on legitimate patients while at the same time effectively eliminating the problem of tampering by diversion, adulteration, or pulverization of the medication for abuse by addicts. The compositions and methods herein are of value to those practiced in the medical arts and simultaneously possess no value or utility to individuals seeking to abuse or profit from the abuse of such analgesics.

It should be understood that in addition to the ingredients particularly mentioned above, the compositions herein may include other agents conventional in the art, having regard to the type of composition in question. For example, agents suitable for oral administration may include such further agents as binders, sweetners, thickeners, flavouring agents, disintegrating agents, coating agents, preservatives, lubricants and/or time delay agents.

The formulation may also contain carriers, diluents and excipients. Details of pharmaceutically acceptable carriers, diluents and excipients and methods of preparing pharmaceutical compositions and formulations are provided in Remmingtons Pharmaceutical Sciences 18^{th} Edition, 1990, Mack Publishing Co., Easton, Pa., USA.

In an embodiment, the active agents may also be presented for use in veterinary compositions. These may be prepared by any suitable means known in the art. Examples of such compositions include those adapted for:
(a) oral administration, e.g. drenches including aqueous and non-aqueous solutions or suspensions, tablets, boluses, powders, granules, pellets for admixture with feedstuffs, pastes for application to the tongue;
(b) parenteral administration, e.g. subcutaneous, intra-articular, intramuscular or intravenous injection as a sterile solution or suspension or through intra-nasal administration;
(c) topical application, e.g. creams, ointments, gels, lotions, etc.

In another embodiment, the active agents are administered orally, preferably in the form of a tablet, capsule, lozenge or liquid. The administered composition may include a surfactant and/or solubility improver. A suitable solubility improver is water-soluble polyethoxylated caster oil and an example of a suitable surfactant is Cremophor EL. Dose ranges suitable for the NK antagonists are, for example, 100 to 1500 mg orally, every six hours including 100, 200, 300, 400, 500, 600, 700, 800, 900, 1000, 1100, 1200, 1300, 1400, 1500. Suitable dose ranges for morphine are 2.5 to 20 mg every 3 to 6 hours such as 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20 and for oxycodone and other opioids 2 to 50 mg every 3 to 12 hours such as 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 31.5, 32, 32.5, 33, 33.5, 34, 34.5, 35, 35.5, 36, 36.5, 37, 37.5, 38, 38.5, 39, 39.5, 40, 40.5, 41, 41.5, 42, 42.5, 43, 43.5, 44, 44.5, 45, 45.5, 46, 46.5, 47, 47.5, 48, 48.5, 49, 49.5, 50 micrograms/hour or by body weight.

In one aspect, fentanyl is administered at a rate and concentration of 100 micrograms/hour.

In another aspect, tramadol is administered at a rate of 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 micrograms/hour or per kg body weight.

In a related aspect an NSAID can be administered at 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 micrograms/hour or per kg body weight.

In a further aspect, a neurosteroid can be administered at 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 micrograms/hour or per kg body weight.

The calcium channel antagonists can be administered without being limited to, a rate of 0, 1, 1, 5, 10, 15, 20, 25, 30, 35, 40, 45, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400 milligrams/hour or per kg body weight.

Mechanical devices are also provided for introduction to or in a body or body cavity coated with a sustained or slow release formulation of an NK antagonist combined with the neuronal excitation inhibitor. Examples of mechanical devices include stents, catheters, artificial limbs, pins, needles, intrathecal implants and the like. Reference to an “intrathecal implant” includes reference to a cylindrical thread or device comprising a semipermeable membrane which permits passage or partial passage of small molecules (such as nutrients ad drugs in and cellular metabolic products out). The implant may also contain genetically modified or cultured cells (including stem cells) which secrete out useful cytokines and other metabolites. The implant may be designed to release molecules (or intake cellular by-products) for days, weeks, months or even years.

Stents, for example, typically have a lumen, inner and outer surfaces, and openings extending from the outer surface to the inner surface. The present invention extends to a method for coating a surface of a stent. At least a portion of the stent is placed in contact with a coating solution containing a coating material to be deposited on the surface of the stent. A thread is inserted through the lumen of the stent, and relative motion between the stent and the thread is produced to substantially remove coating material within the openings.

The thread can have a diameter substantially smaller than the diameter of the lumen. The thread can be inserted through the lumen either after or prior to contacting the stent with the coating solution. Relative motion between the stent and the thread can be produced prior to contacting the stent with the coating solution to clean the stent. The thread can be either a filament or a cable with a plurality of wires. The thread can be made of a metallic or polymeric material.

The stent can be dipped into the coating solution or spray coated with the coating solution. The coating material can include a biocompatible polymer, either with or without a pharmaceutically active compound.

In one embodiment, the relative motion is oscillatory motion produced by a vibrating device. The oscillations can be changed (magnitude and/or frequency) to vary thickness of the coating solution on the stent. In another embodiment, the relative motion is produced by a shaker table. Regardless of the type of motion, the relative motion can be produced either after or while the stent is in contact with the coating solution.

The relative motion between the stent and the thread can include initially moving the stent in a horizontal direction substantially parallel to the length of the thread and subsequently moving the stent in a vertical direction substantially perpendicular to the length of the thread. The movement in the horizontal direction can be repeated, with pauses between repetitions. The movement in the vertical direction can also be repeated, with the horizontal and vertical movements alternating.

In order to smooth the relative motion, the thread can be coupled to a damping compensator. The damping compensator connects the thread to a vibrating device. In one embodiment, the damping compensator comprises first and second filaments connected to the thread.

The relative motion can be motion of the stent along the thread. For example, a first end of the thread can be attached to a first stand at a first height and a second end of the thread is attached to a second stand at a second height. The relative motion is produced by a gravity gradient, with the first height differing from the second height. Furthermore, the stent can be moved back and forth between the first and second stands by sequentially increasing or decreasing at least one of the first and second heights. In this way, multiple coatings can be applied to the stent.

The relative motion can also be rotation of the stent relative to the thread. A stream of gas can be passed along at least a portion of the surface of the stent to rotate the stent relative to the thread. The rotation can also occur in conjunction with other relative motion between the stent and the thread.

An implantable medical device is also provided having an outer surface covered at least in part by an NK antagonist and a neuronal excitation inhibitor or pharmaceutically acceptable salts, derivative, homolog or analog thereof and optionally an opioid and/or other active agent, a conformal coating of a hydrophobic elastomeric material incorporating an amount of active material therein for timed delivery therefrom and means associated with the conformal coating to provide a non-thrombogenic surface after the timed delivery of the active material.

In an embodiment, the conformal coating comprises an amount of finely divided biologically active material in the hydrophobic elastomeric material.

The following examples are intended for the purpose of illustration only and are not intended to limit the generality of the methods, compositions, protocols and devices as herein described.

### Example 1

### The Use of NK1 Antagonists in the Treatment of Pain

Experiments were performed on male Wistar rats (wt 150-220 g). Three series of experiments were performed in an observer blinded fashion with saline (negative) controls and GABAPentin (positive) controls. The investigations were in three stages:
1. a range of doses of aprepitant (an example of a NK antagonist) given alone and in combination with flupiritine (an example of an inhibitor of neuronal excitation) were tested for sedating effects using the open field activity monitor. In this way it was determined which doses of drug and drug combinations could be used to test for analgesic effects without causing sedation;
2. a range of doses of aprepitant given alone and in combination with flupirtine were tested for the ability to reverse the allodynia effect causes by intraplantar injection of carrageenan;
3. a range of doses of aprepitant given alone and in combination with flupirtine were tested for the ability to reverse the allodynia and hyperalgesia caused by streptozotocin-induced diabetic neuropathy.

### Open Field Activity Monitor

Individual rats (several rats per treatment group) were placed in an open field activity monitor in which the movement of the rat could be monitored remotely by the frequency and number of interruption of infrared beams directed across the box in a grid. The activity in the monitor was measured for periods of 20 minutes in each rat. This was performed in groups of rats that received (1) control injection of saline, (2) GABAPentin 50 mg/kg as the maximum dose of the drug found previously not to be sedating (drug positive control) administered intraperitoneally, or (3) after pharmacological interventions that involve the administration of aprepitant and flupiritine given at a range of doses alone and in combination. If a rat was sedated by a drug or drug combination, the movements recorded were less. Since rates became habituated to the open field monitor, only one experiment was performed on each rat with this test.

The results of these experiments are shown in Tables 1 and 2. It was concluded from these experiments that aprepitant alone caused no sedating effects up to and including the dose of 6.25 mg/kg and dose of aprepitant up to 3.12 mg/kg could be used in combination with flupirtine 10 mg/kg without causing sedation. These were the upper dose limits for subsequent experiments investigating the analgesic effects of these drugs in administered together in combination.

### Carrageenan Paw Inflammation

Inflammation involves the release of a number of substances into the tissues following injury. These substances including prostaglandins, bradykinin, inflammatory peptides such as substance P and calcitonin gene related peptide and also a number of cytokines. The carrageenan paw inflammation model involves induction of inflammation and oedema in one paw of the rat by the intraplantar injection of carrageenan (6 mg per 150 g1). This is a single intraplantar injection using a fine needle and syringe whilst restraining the rat gently. The rats were then subjected to nociceptive threshold measurement using withdrawal from stimulation with Von Frey hairs (measures allodynia). Nociceptive thresholds were measured in groups of rats prior to the intraplantar injection. The measurements were continued three hours after the intraplantar injection when the inflammation had developed. At that stage allodynia had developed. Each rat then received an intraperitoneal injection of a dose of aprepitant or flupirtine alone or both drugs in combination or a control. Measurements of the allodynia nociceptive thresholds for the next one hour were then used to assess the antinocicepetive effect of the dose of the single drugs, drug combination or control. Dose response curves for the test drugs and controls were plotted as means ±standard errors of the mean of the replicates at each dose.

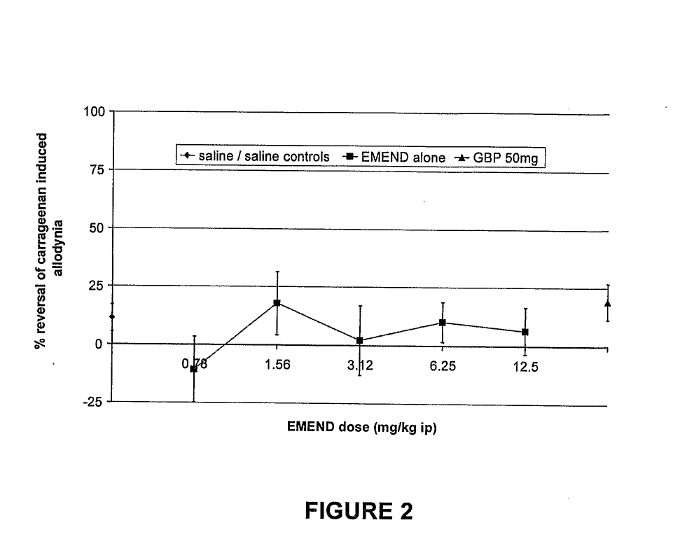

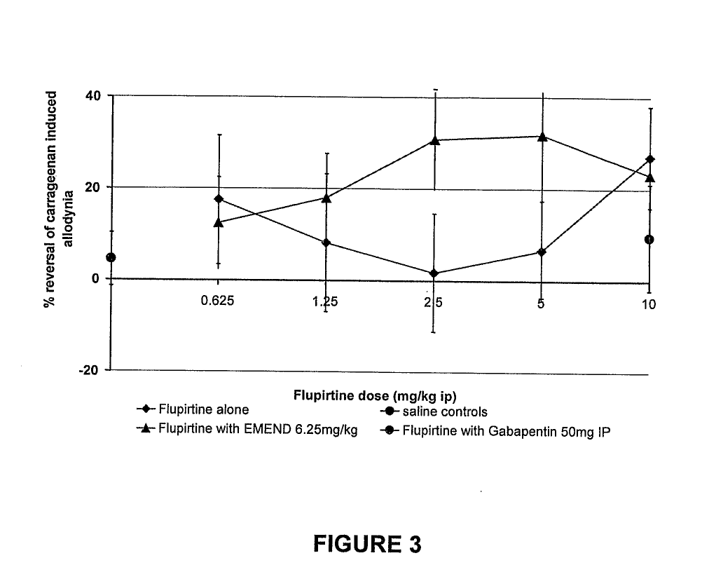

The results of these experiments are shown in FIGS. 2 and 3. It can be seen that neither flupirtine or aprepitant given at non-sedating doses caused any antinociception in this test when these compounds were administered alone. However, when they were administered in combination there was synergy; the combination of flupiritine 2.5 mg/kg with aprepitant 6.25 mg/kg caused a significant analgesic effect which was greater than saline or GABAPentin controls. There was an absence of an analgesic effect when GABApentin and aprepitant were combined.

TABLE 1
results of experiments testing for sedation in the open field activity monitor

| | Saline | Aprep | Aprep | Aprep | Aprep | Aprep |
|---|---|---|---|---|---|---|
| | control | 6.25 mg/kg | 12.5 mg/kg | 25 mg/kg | 50 mg/kg | 100 mg/kg |
| | IP | IP | IP | IP | IP | IP |
| mean | 804.2 | 869.0 | 915.0* | 964.6* | 976.6* | 1058.2* |
| SD | 93.5 | 100.5 | 59.3 | 95.7 | 89.3 | 46.3 |
| n | 20 | 10 | 10 | 10 | 12 | 5 |

*p < 0.01 One way Anova comparison with saline controls

TABLE 2
results of experiments testing for sedation
in the open field activity monitor

| | | Aprepitant | |
|---|---|---|---|
| | Saline | 6.25 mg + | GBP 50 mg + |
| | control IP | Flupirtine 10 mg IP | Flupirtine 10 mg IP |
| mean | 1375.0 | 1574.7* | 1543.3* |
| SD | 95.7 | 63.2 | 128.8 |
| n | 11 | 9 | 11 |

*p < 0.01 One way Anova Comparison with saline controls

### Example 2

### Screening of Compounds in an In Vivo Model

The animal experiment that is used as a model for pain in humans caused by inflammation is carrageenan paw inflammation in rats. Intraplantar injection of carrageenan into the footpad of one hind paw in male Wistar rats causes inflammation and swelling of the hind paw over a period of three hours. This leads to a reduction in paw pressure withdrawal thresholds. Nociceptive thresholds are measured in the hind paw of normal rats and in the inflamed hind paw of carrageenan-treated rats using measurements of paw withdrawal from a noxious stimulus such as heat—the paw flick test; alternatively paw withdrawal from pressure exerted on the inflamed paw by calibrated von Frey hairs may be used as the stimulus. The essential points demonstrated by these results are:
1. The maximum non-sedating dose of each drug is first determined by giving a range of doses to rats followed by assessment of sedation using two tests: open field exploration and the rota-rod test.
2. Using carrageenan paw inflammation we show that each compound when used alone causes none or a partial antinociceptive (analgesic) effect even when the largest non-sedating dose is used.
3. Using carrageenan paw inflammation we show that an NK1 antagonist administered to rats in combination with another compound such as flupirtine or opioid causes much greater antinociception than can be achieved with the maximal non-sedating doses of either drug when administered alone.
4. Using the open field activity and rota-rod tests we show that the combinations of drugs that cause the increased antinociception described in paragraph 3 above, do not cause sedation thus showing that the two compounds interact in causing better pain relief but not to cause increased sedation.

### Methods

General methods and guidelines for investigation of experimental pain in conscious animals were described in Zimmerman Pain, 16:109-110, 1983. Experiments are performed on male Wistar rats (140-250 g). All antinociceptive drugs used are administered via the intraperitoneal route (ip). Experiments are performed in a blinded manner i.e. the person performing the nociceptive testing is unaware of the drug administered to each animal. Nociceptive thresholds are measured using noxious heat, von Frey hair or paw pressure withdrawal in the hind paw of rats inflamed by intraplantar injection of carrageenan. Animals are not used for multiple experiments.

### Assessment of Sedation

The maximum dose of each compound that does not cause sedation is determined prior to testing for antinociceptive properties. This is done so that the results observed with the nociceptive testing paradigms are indeed due to an antinociceptive effect and not sedation or inattention.

### Rotarod Test

The rats are naïve to the drugs with no previous exposure to the rotarod test. They are placed on the rotarod accelerator treadmill (7650 accelerator rotarod, Ugo Basile, Italy) set at the minimum speed for two training sessions of 1-2 minutes separated by an interval of 30-60 minutes. After this conditioning period the intraperitoneal injection of vehicle, drug, or drug combination is given. Five minutes later the animals are placed onto the rotarod at a constant speed of 4 revolutions per minute. As the animal takes grip of the drum the accelerator mode is selected on the treadmill, i.e. the rotation rate of the drum is increased linearly at the rate of 20 revolutions per minute every minute thereafter. The time is measured from the start of the acceleration period until the rat falls off the drum; this is the control (pre-treatment) performance time for each rat. A cut-off or maximum runtime for the test is 2 minutes because normal non-sedated rats all run for 2 minutes at which time the test is terminated. This test is performed on each rat at intervals of 10 minutes between each run for 30-60 minutes. These values are combined for each drug at each dose to calculate means ±SEM.

### Open Field Activity Monitor

Rats are observed in a commercially available open-field arena in which locomotor and exploratory activity can be monitored in darkness by the breaking of infrared beams arranged in a grid pattern over the entire area (MedAssociates Inc. St. Albans, Vt., USA 05478). The observations are started 5 minutes after the intraperitoneal administration of drugs. The total time of observations in all cases is 20 minutes. In order to avoid habituation to the activity monitor, animals are used once in this test for sedation. Rest time is defined as the time spent with no new infrared beam interruptions.

Each set of experiments is performed with matching controls. The data from vehicle-treated controls are compared with the data following drug injections using one-way ANOVA with Tukey Kramer post hoc test. These comparisons allow definition of drug doses that cause sedation.

### Induction Of Inflammation By Intraplantar Carrageenan Injection

Experimental inflammation of the right hind paw is induced by an intraplantar injection of 100 μl 2% carrageenan diluted in saline (Sigma-Aldrich Pty. Ltd. Australia). Two and a half hours are allowed for the induction of inflammation (Greizerstein Subst Alcohol Actions Misuse 4(6):393-9, 1983; Honmura et al. Lasers Surg Med 13(4):463-9, 1993; Meller et al. Neuroscience 60(2):367-74, 1994).

### Measurement of Paw Withdrawal Latency

The Basile Plantar Test (Hargreaves' Method) enables the researcher to discern a peripherally mediated response to thermal stimulation caused by drugs in unrestrained rats. It basically consists of a movable infrared generator, which the operator glides below a glass pane upon which the rats stand in a 3-compartment Perspex enclosure. A controller, via a suitable sensor placed in the infra-red generator drum, detects the withdrawal latency of the animal paw in 0.1 second steps.

Paw withdrawal thresholds may also be assessed with von Frey hairs clibrated to different pressures, the withdrawal threshold being the minimum force applied to the paw that causes the rat to withdraw it's paw from the stimulus.

### Nociceptive Paradigm

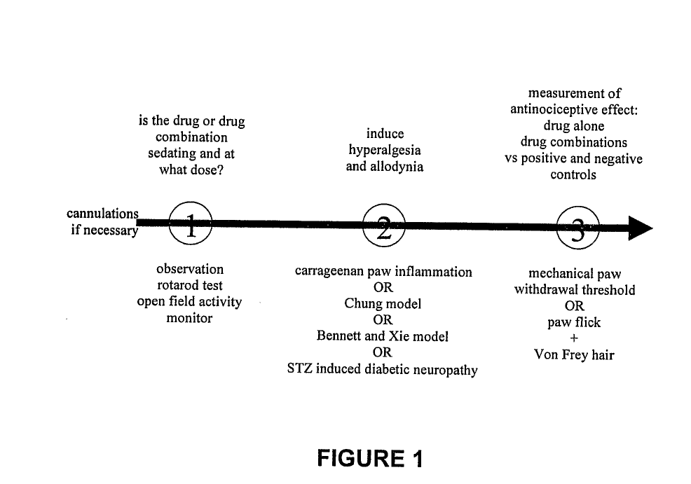

Paw withdrawal latencies or thresholds are measured in each individual rat before the induction of inflammation with carrageenan injections every 10 minutes until 3 stable readings are attained. After two and a half hours, once inflammation is established, paw thresholds are again measured; three readings at ten minute intervals. A test drug or vehicle is injected and paw flick latencies or mechanical (von Frey) withdrawal thresholds are measured at 10-minute intervals for the following 40-60 minutes. The protocol is shown in FIG. 1.

Experiments are performed for a range of non-sedating doses of the test neurokinin antagonist compounds given alone and in combination with other compounds such as flupirtine, retigabine, NSAID's and opioids; all being performed in a blinded manner with vehicle controls.

Those skilled in the art will appreciate that the invention described herein is susceptible to variations and modifications other than those specifically described. It is to be understood that the invention includes all such variations and modifications. The present invention also includes all of the steps, features, compositions and compounds referred to, or indicated in this specification, individually or collectively, and any and all combinations of any two or more of said steps or features.

### BIBLIOGRAPHY

Bundgaard Design of prodrugs, Elsevier, 1985
Greizerstein Subst Alcohol Actions Misuse 4(6):393-9, 1983
Grimwood et al. Molecular Pharmacology 4:923 1992
Honmura et al. Lasers Surg Med 13(4):463-9, 1993
Jacobson et al. J Pharmacol Exp Ther 110.243, 1987
Leeson P. D. Drug Design For Neuroscience 13:338-381, 1993
Mayer et al. J Neurophysiol 645, 1988
Meller et al. Neuroscience 60(2):367-74, 1994
Olney & Farber, Neuropsychopharmacology 13:335, 1995
Remmingtons Pharmaceutical Sciences 18^{th} Edition, Mack Publishing Co., Easton, Pa., USA, 1990
Sawynok et al. Pain 80:45, 1999
Sawynok et al. Pain 82.149, 1999
Thurkauf et al. J Med Chem 31:2257, 1988,
Yoneda et al. J Neurochem 62:102, 1994
Zimmennan Pain 16:109-110, 1983

1. A method for inducing an analgesic response to inflammatory pain without inducing overt sedation in a mammal comprising administering to the mammal an amount of an neurokinin (NK) antagonist in combination with a neuronal excitation inhibitor, which combination is effective in reducing the level of or otherwise ameliorating the sensation of pain associated with inflammatory processes.

2. The method of claim 1 wherein the NK antagonist is an NK1 antagonist.

3. The method of claim 2 wherein the NK1 antagonist is aprepitant.

4. The method of claim 1 wherein the neuronal excitation inhibitor is flupirtine or a pharmaceutically acceptable salt thereof.

5. The method of claim 1 wherein the neuronal excitation inhibitor is retigabine or a pharmaceutically acceptable salt thereof.

6. The method of claim 1 wherein the neuronal excitation inhibitor is a potassium channel opener.

7. The method of claim 1 wherein the neuronal excitation inhibitor is an opioid or is a pharmaceutically acceptable salt, derivate, homolog or analog thereof.

8. The method of claim 4 wherein the neuronal excitation inhibitor is an NMDA antagonist.

9. The method of claim 1 wherein the neuronal excitation inhibitor is modulator of TRPV1 receptor.

10. The method of claim 4 wherein flupirtine is administered in an amount of about 0.25 mg/kg to about 20 mg/kg of body weight.

11. The method of claim 1 wherein the mammal is human.

12. The method of claim 1 further comprising the steps of selecting a mammal on the basis of the mammal having symptoms of inflammatory pain.

13. A delivery system for inducing an analgesic response in a mammal having inflammatory pain said delivery system comprising combined or separate formulations of (1) an NK antagonist; (2) an neuronal excitation inhibitor; and optionally (3) one or more further active agents.

14. The delivery system of claim 13 wherein the NK antagonist is an NK1 antagonist.

15. The delivery system of claim 13 wherein the NK1 antagonist is aprepitant.

16. The delivery system of claim 13 wherein the neuronal excitation inhibitor is flupirtine or a pharmaceutically acceptable salt thereof.

17. The delivery system of claim 13 wherein the neuronal excitation inhibitor is retigabine or a pharmaceutically acceptable salt thereof.

18. The delivery system of claim 13 wherein the neuronal excitation inhibitor is a potassium channel opener.

19. The delivery system of claim 13 wherein the neuronal excitation inhibitor is an opioid or a pharmaceutically acceptable salt, derivative, homolog or analog thereof.

20. The delivery system of claim 13 where in the neuronal excitation inhibitor is an NMDA antagonist.

21. The delivery system of claim 13 wherein the neuronal excitation inhibitor is a calcium channel antagonist.

22. The delivery system of claim 13 wherein the neuronal excitation inhibitor is an NSAID.

23. The delivery system of claim 13 wherein the neuronal excitation inhibitor is a modulator of TRPV1 receptor.

24. The delivery system of claim 16 wherein flupirtine is administered in an amount of about 0.25 mg/kg to about 20 mg/kg of body weight.

25. A method of treating inflammatory pain associated with a disease or physiological condition in a mammal without inducing overt sedation, said method comprising administering to said mammal an effective amount of an NK antagonist and a neuronal excitation inhibitor.

26. The method of claim 25 wherein the NK antagonist is an NK1 antagonist.

27. The method of claim 25 wherein the NK1 antagonist is aprepitant.

28. The method of claim 25 wherein the neuronal excitation inhibitor is flupirtine or a pharmaceutically acceptable salt thereof.

29. The method of claim 25 wherein the neuronal excitation inhibitor is retigabine or a pharmaceutically accepted salt thereof.

30. The method of claim 25 wherein the neuronal excitation inhibitor is a potassium channel opener.

31. The method of claim 25 wherein the neuronal excitation inhibitor is an opioid or a pharmaceutically acceptable salt, derivative, homolog or analog thereof.

32. The method of claim 25 where in the neuronal excitation inhibitor is an NMDA antagonist.

33. The method of claim 25 wherein the neuronal excitation inhibitor is a calcium channel antagonist.

34. The method of claim 25 wherein the neuronal excitation inhibitor is an NSAID.

35. The method of claim 25 wherein the neuronal excitation inhibitor is a sodium channel blocker.

36. The method of claim 25 wherein the neuronal excitation inhibitor is a modulator of TRPV1 receptor.

37. The method of claim 28 wherein flupirtine is administered in an amount of about 0.25 mg/kg to about 20 mg/kg of body weight.

38. The method of claim 25 wherein the disease is selected from acne, angina, arthritis, aspiration pneumonia, disease, empyema, gastroenteritis, inflammation, intestinal flu, NEC, necrotizing enterocolitis, pelvic inflammatory disease, pharyngitis, PID, pleurisy, raw throat, redness, rubor, sore throat, stomach flu and urinary tract infections, Chronic Inflammatory Demyelinating Polyneuropathy and post-operative pain and Chronic Inflammatory Demyelinating Polyradiculoneuropathy.

39. A system for the controlled release of active compounds selected from an NK antagonist and a neuronal excitation inhibitor or a pharmaceutically acceptable salt, derivative, homolog or analog thereof, wherein the system comprises:
(a) a deposit-core comprising an effective amount of a first active compound and having defined geometric form, and
(b) a support-platform applied to the deposit-core, wherein the support-platform contains a second active compound, and at least one compound selected from the group consisting of:
(i) a polymeric material which swells on contact with water or aqueous liquids and a gellable polymeric material wherein the ratio of the swellable polymeric material to the gellable polymeric material is in the range 1:9 to 9:1, and
(ii) a single polymeric material having both swelling and gelling properties, and wherein the support-platform is an elastic support applied to the deposit-core so that it partially covers the surface of the deposit-core and follows changes due to hydration of the deposit-core and is slowly soluble and/or slowly gellable in aqueous fluids.

40. A system for the controlled release for an NK antagonist and a neuronal excitation inhibitor wherein the system comprises:
(a) a deposit-core comprising an effective amount of (1) an NK antagonist and (2) a neuronal excitation inhibitor form; and
(b) a support platform applied to the deposit-core, the support platform comprising at least one compound selected from the group consisting of:
(i) a polymeric material which swells on contact with water or aqueous liquids and a gellable polymeric material wherein the ratio of the swellable polymeric material to the gellable polymeric material is in the range 1:9 to 9:1, and
(ii) a single polymeric material having both swelling and gelling properties, and wherein the support-platform is an elastic support applied to the deposit-core so that it partially covers the surface of the deposit-core and follows changes due to hydration of the deposit-core and is slowly soluble and/or slowly gellable in aqueous fluids.

41. A system for the controlled release of claim 39 wherein the support platform comprises a hydroxypropylmethyl cellulose.

42. A system for the controlled release of claim 39 wherein the support platform comprises a plasticizer, a binder, a hydrophilic agent and a hydrophobic agent.

43. A method of treatment of a subject said method comprising selecting a subject on the basis of symptoms of inflammatory pain and administering to said subject an NK antagonist and a neuronal excitation inhibitor wherein the treatment does not cause overt sedation.

44. The method of claim 43 wherein the subject is a human.

45. A system for the controlled release of claim 40 wherein the support platform comprises a hydroxypropylmethyl cellulose.

46. A system for the controlled release of claim 40 wherein the support platform comprises a plasticizer, a binder, a hydrophilic agent and a hydrophobic agent.
